(19) United States
(12) Patent Application Publication
(10) Pub. No.: US 2009/0050159 A1
(43) Pub. Date: Feb. 26, 2009

(54) Arm support device and methods of use thereof

(76) Inventors: Daniel L. Jamerson, Lakeside, CA (US); Kimberly Ann Jamerson, Lakeside, CA (US)

Correspondence Address: GORDON & REES LLP, 101 WEST BROADWAY, SUITE 1600, SAN DIEGO, CA, 92101, US

(21) Appl. No.: 11/895,349

(22) Filed: Aug. 24, 2007

(51) Int. Cl. A61F 5/00; A61F 5/01

(52) U.S. Cl. 128/845; 602/4

(57) ABSTRACT

According to one embodiment, an arm support device includes a sack of flexible and stretchable material. The sack is open at one end and closed (or closeable) on all other sides. The user inserts both arms into the sack and pulls the sack over the upper arms and shoulders. With the arms thus inserted, the user is free to relax and allow the stretch of the sack to provide arm support. In some embodiments, the sack has no other openings besides the top end, and in these embodiments the arm support device doubles as a travel bag for carrying small items.

### FIELD OF THE INVENTION

The present invention relates generally to travel accessories, specifically devices that support the arms of travelers.

### BACKGROUND OF THE INVENTION

Modern travel often requires travelers to be seated very close to strangers for long periods of time. For example, most modern travel takes place in airplanes, trains, buses, or other vehicles in which the seats are extremely close together. Frequently the seats are so close together that only one armrest is provided for two adjoining seats. Often a person in one of these seats monopolizes the shared armrest, leaving no room on the armrest for the person in the adjoining seat.

One solution to this problem is for the traveler to use his own body as an armrest by folding his arms across his chest or stomach. However, it is uncomfortable to maintain this position for any length of time. Furthermore, it is difficult to sleep in this position. A traveler who falls asleep in this position is likely to have his arms become unfolded and fall onto the armrest, possibly onto the seatmate's arm. Accordingly, even a courteous traveler who attempts to sleep in the arms-folded position may unintentionally invade the personal space of the person in the next seat.

The difficulty of keeping one's arms in a comfortable position in tight seating arrangements is thus a major drawback to modern travel. An attempt to solve this problem is found in U.S. Pat. No. 6,435,185. However, the device disclosed therein requires the traveler to place the device around his neck and over his shoulders. This can be difficult to accomplish while seated in a small seat without disturbing one's neighbors.

Another attempt to solve this problem is found in U.S. Pat. No. 6,966,069. A disadvantage of this device is that it requires the traveler to pull the device over her head, thus possibly disturbing her seatmates. A second disadvantage of this device is that the position of the user's arms are restricted to the location of the interior pocket.

Accordingly, there is a need for an arm supporting travel device that can be used by a traveler without disturbing the traveler's seatmates and that allows the traveler to comfortably support the arms in multiple positions.

### SUMMARY OF THE INVENTION

It is an object of the present invention to provide an arm support device that addresses this need.

According to one embodiment, an arm support device includes a sack of flexible and stretchable material. The sack is open at one end and closed (or closeable) on all other sides. The user inserts both arms into the sack and pulls the sack over the upper arms and shoulders. With the arms thus inserted, the user is free to relax and allow the stretch of the sack to provide arm support. In some embodiments, the sack has no other openings besides the top end, and in these embodiments the arm support device doubles as a travel bag for carrying small items.

In other embodiments of the invention, pockets are provided on the front or back of the arm support device, either on an inside or outside surface. Pull tabs may also be provided near the top edge of the sack to facilitate pulling it up. Also optional is a hole or slit on the front of the sack that may be closeable with a fastener. With the user's arms inserted into the sack, the user can reach through the slit with one or both hands to access external pockets or to reach other items. A pocket on the front or back of the sack may be adapted to store the wadded arm support device so that, while not in use, it is self-contained in a small package.

In other embodiments, the arm support device may optionally include adjustment straps by which the user can adjust the degree of tension in the sack and thus the amount of arm support it provides. Furthermore, to increase functionality as a travel bag, a drawstring may be sewn into the sack near the open end. The open end of the sack may then be closed by cinching the drawstring.

In another embodiment of the arm support device, an optional neck strap is attached to the top edge of the sack to help keep it in place on the user's upper arms and shoulders. This neck strap may be stuffed with batting or similar material so that the neck strap can also be used as pillow. To further increase the comfort of the user, a blanket may be attached to the bottom edge of the arm support device. The blanket may optionally include pockets, and the arm support device may optionally include a pocket for storing the blanket when not in use.

Another embodiment of the arm support device may incorporate optional ventilation openings. These ventilation openings may be on the front face, the rear face, or both.

An arm support device according to yet another embodiment may optionally include an internal divider so that the sack is divided into two separate compartments. The internal divider may include a slit so that the compartments are connected.

Another embodiment of the arm support device includes an area on the front face for indicia such as advertising.

Also contemplated by the present invention is a method of supporting the arms of a person. The person inserts his arms into the arm support device and pulls it up to his shoulders. The person may then cross his arms over his torso and then relax to allow the walls of the arm support device to cradle his arms. The arm support device may be held in place by friction alone, or by friction supplemented by a neck strap.

### BRIEF DESCRIPTION OF THE DRAWINGS

FIG. 1 is a front elevation view of an arm support device according to a first exemplary embodiment.

FIG. 2 is a back elevation view of the arm support device of FIG. 1.

FIG. 3 is a front elevation view of an arm support device according to a second exemplary embodiment.

FIG. 4 is a back elevation view of an arm support device according to a third exemplary embodiment.

FIG. 5 is a front elevation view of an arm support device according to a fourth exemplary embodiment.

FIG. 6 is a top plan view of an arm support device according to a fifth exemplary embodiment.

FIG. 7 is a front elevation view of the arm support device of FIG. 6.

FIG. 8 is a front elevation view of an arm support device according to a sixth exemplary embodiment.

FIG. 9 is a front elevation view of an arm support device according to a seventh exemplary embodiment.

FIG. 10 is a back elevation view of the arm support device of FIG. 9.

### DETAILED DESCRIPTION

Exemplary embodiments of an arm support device are described in detail below with reference to the appended figures, wherein like elements are referenced with like numerals throughout. The figures are not necessarily drawn to scale and do not necessarily show every detail or structure of the various embodiments of the invention, but rather illustrate exemplary embodiments and features in order to provide an enabling description of such embodiments.

Occasionally throughout this specification, the user of the arm support device may be referred to as a “traveler.” It is to be understood, however, that the arm support device can be used by anyone wishing to comfortably support their arms, whether or not the user is traveling at the time.

An arm support device basically comprises a flexible and stretchable sack. The top end of the sack is open while the bottom end and the sides are closed or closeable with fasteners. The open top end allows a traveler to insert her arms until the upper edge meets her underarms. With her arms thus fully inserted, the traveler folds her arms across her torso in a comfortable position. The inside surfaces of the arm support device contact and support the traveler's arms as it stretches. This support prevents the traveler's arms from slipping off her torso and allows her to effortlessly rest in a comfortable position. Additionally, in some embodiments, the arm support device has no non-closeable openings, and can thus carry small items placed inside it. Thus, in these embodiments, the arm support device can be used as a convenient travel bag when not in use as an arm support.

As used throughout this specification, the top of the arm support device is the open end into which users insert their arms. The bottom end is the closed end opposite the top edge. Furthermore, the length of the arm support device is the distance from its top edge to its bottom edge. The perimeter and the width of the arm support device, unless otherwise indicated, are measured at the top edge. The front face of the arm support device is the exterior surface of the arm support that faces away from the user during use. The rear face is opposite the front face. Additionally, the arm support device is considered fully stretched when application of additional stretching force does not yield any appreciable amount of further stretching. The arm support is considered completely unstretched when no stretching force is applied at all. A stretching force is any force that causes a dimension of the arm support to increase in length.

One benefit of the arm support device is that it is a simple way for travelers to comfortably support their arms in a wide variety of positions. Unlike other arm supporting devices or garments, which restrict the user's arms to one position, the present invention allows the user to move his arms into many different comfortably supported positions, so long as the user's arms are inside the sack and folded across his torso.

Another advantage of the arm support device is that it is easy to use in close quarters. Unlike an arm supporting garment that must be pulled over the user's head like a shirt, the arm support device is pulled over the arms and partially over the shoulders, but not over the head. A traveler in a tight seating situation may find it exceedingly difficult to pull an arm supporting garment over his head without elbowing or otherwise disturbing his seatmate. By contrast, a traveler can pull on the arm support device of the present invention without raising his arms above shoulder level. Accordingly, a person can use the arm support device in tight seating arrangements without disturbing any seatmates.

Another benefit of the arm support device is that it provides arm support in multiple directions. The lower portion of the arm support device provides generally vertical support for the user's arms and hands, preventing them from falling into her lap. The central, upper, and side portions of the arm support device provide lateral support for the user's arms, preventing them from falling to her sides. These multiple directions of support allow the user a wider range of comfortable arm positions.

A further advantage of the arm support device is that it is very comfortable. Not only are the user's arms supported in a comfortable position, the arm support device also provides warmth and a feeling of coziness.

Yet another benefit of the arm support device is that, in some embodiments, it is a travel bag in addition to an arm support device. Arm supporting garments with head/neck openings cannot be used as bags because items placed inside would fall out of the head/neck opening. In some embodiments, the arm support device of the present invention has no such opening and thus is capable of being used as a bag. Although the arm support device may have a slit on the front face of the sack, the slit is not near the bottom of the sack and may be closeable with a fastener. Thus, items placed inside will not fall out through the bottom.

An important feature of the arm support device is its stretch factors. As used herein, the term “stretch factor” shall refer to a ratio equal to the length of a given dimension of the fully-stretched arm support divided by the length of that same dimension when the arm support is completely unstretched. For example, the stretch factor of the length of the arm support device (SF_{L}) is equal to the ratio of fully stretched length to the completely unstretched length. Likewise, the stretch factor of the perimeter of the arm support device (SF_{P}) is equal to the ratio of the fully stretched perimeter to the completely unstretched perimeter.

The stretch factor of the arm support device is an important parameter because the arm support cannot be dimensioned properly and/or it cannot provide comfortable arm support if the stretch factor is outside of a well-defined range. Material with excessive elasticity (stretch factor too high) provides insufficient arm support: as a user's arms fall to her sides, the material simply stretches out and the user's arms come to rest just as they would if the arm support was not used at all.

If, on the other hand, the material of the arm support is insufficiently elastic (stretch factor too low), the arm support cannot be dimensioned properly. If an unstretched arm support made from insufficiently elastic material is dimensioned large enough to fit around the user's shoulders, the material will not remain frictionally held in place on the user's upper arms and shoulders. Instead, it will tend to slide down to the user's elbows where it cannot provide sufficient arm support. If, on the other hand, an unstretched arm support made from insufficiently elastic material is dimensioned small enough to tightly fit around the user's upper arms and shoulders, it will be too small for the user to be able to fully fold her arms across her torso. Accordingly, the stretch factors of the arm support must fall within a range such that an arm support dimensioned to properly fit the user will simultaneously be held in place by friction and also provide arm support.

The arm support device has two relevant stretch factors: the stretch factor of its length (SF_{L}) and the stretch factor of its perimeter (SF_{P}). Of these, the second is more critical than the first. The SF_{P} value is measured at the top edge, and is equal to the fully stretched perimeter divided by the completely unstretched perimeter. In an exemplary embodiment, SF_{P} is approximately equal to 1.5, and in other embodiments should be between about 1.25 and 2.0. However, in alternative embodiments, SF_{P} may be anywhere from 1.0 to 2.5.

The SF_{P} value is critical because it controls both how much lateral arm support the arm support device provides, as well as the amount of friction between the arm support device and the user's arms. The perimeter stretch factor must be high enough to allow the user's arms to begin to slide from the folded position toward her sides, but low enough to fully support her arms before they fall completely to her sides. However, if the perimeter stretch factor is too low, the arm support device cannot be dimensioned to properly fit the intended user, as explained above.

The SF_{L} value of the arm support device is less critical, but still relevant. If the arm support device is too elastic (stretch factor too high) in this direction, it will not provide vertical support for the user's arms and hands. However, this does not greatly impair the functionality of the arm support because the user's lap can provide vertical support for the user's hands. If the arm support device is too inelastic in this direction, it will provide vertical support, but it will not be as comfortable as if it were more elastic. Again, although the arm support device is less comfortable if it is inelastic lengthwise, it can still provide lateral arm support so long as the SF_{P} value is within the acceptable range. In an exemplary embodiment, SF_{L} is approximately 1.2, and in other embodiments between about 1.0 and 2.5. It is to be understood, however, that SF_{L} can be any value so long as SF_{P} is within the acceptable range.

Proper dimensioning of the arm support device is important, because if it is too large it will tend to fall off the user's upper arms and shoulders, and if it is too small the user's folded arms will not fit comfortably inside. For this reason, the arm support device comes in a wide range of sizes designed to accommodate users of all sizes. The width of the arm support device is approximately equal to the width of the intended user's shoulders. The length of the arm support device is approximately equal to the distance from the intended user's shoulders to the intended user's elbows. The length, width and perimeter of the arm support will vary depending on the size of the intended user so that it will fit users of any size.

The relative proportions of the arm support device are also important to its functionality. As used herein, the shape factor of the arm support device is defined as the ratio of its width to its length. In an exemplary embodiment, the shape factor is about 1.05, and should be between about 0.8 and 1.3 for optimum functionality. However, the shape factor may be anywhere from about 0.5 to 2.0 in various embodiments.

The arm support device may be made from any flexible, stretchable material, so long as the material allows the arm support to have acceptable stretch factors. More particularly, and without limitation, the arm support device may be made from jersey, fleece, flannel, polyester-cotton blends, and combinations thereof. To create the arm support device, two approximately equal-size panels of fabric may be sewn together on all edges except the top. Alternatively, the arm support may be made from a single panel of fabric that is sewn to itself to form the desired shape. Also alternatively, the front face of the arm support may alone comprise two panels of fabric sewn together, leaving a small slit where they meet, which serves as a hand-access slit. It is to be understood, however, that any number of panels or pieces of material may be used, so long as the resulting shape, dimensions, and stretch factors are proper.

To use the arm support device, a user places it in his lap, with the front face facing up. The user then inserts each arm into the open top end and crosses or folds his arms upon insertion. Grasping the top edge of the sack with each hand, the user pulls the arm support device up and over the upper arms until the top edge is over the top of the user's shoulders and also contacting the user's underarms. Once pulled up as far as possible, the user releases the top edge from his grasp and folds his arms across his torso. When the user relaxes his arm muscles, his arms will begin to fall to his sides. However, as the user's arms begin to fall, the arm support device begins to stretch. The arm support device provides arm supporting forces that are approximately proportional to the amount the material has stretched. At some point the support provided is greater than the weight of the user's arms, which are thus fully supported.

While in use, the arm support device is held in place by friction between its surfaces and the user's arms, shoulders, underarms, and chest. Of particular importance is the friction between the inside surfaces of the upper portion of the arm support and the user's upper arms and shoulders. As the tensile forces in the material increase as the arm support is stretched out, the frictional force holding the snug in place also increases.

The arm support device may include a slit on the front face which allows the user to reach outside of the arm support while using it. Thus, a user need not take off the arm support in order to reach a nearby item. Furthermore, the user can reach outside to hold a book, eat a snack, or drink some water, all without taking off the arm support device. The arm support device may also include pockets or fabric loops on the front face in which the user can conveniently store items such as cell phones, music players, eyeglasses, cosmetics, etc. The user can access items in these pockets or loops through the slit in the front face, without taking off the arm support device. Furthermore, one of the external pockets can be used to store the arm support device when it is not in use. The arm support device is balled up and stuffed inside the pocket, so that it takes up very little space when not in use.

Because the bottom end of the arm support is closed or closeable with a fastener, it can be used as a convenient bag, both while in use as an arm support and while not so in use. While serving as an arm support, the arm support can hold objects that are too big to fit in a pocket, but which the user wishes to have nearby, such as a book for example. While not being used to support the user's arms, the arm support device can hold a large number of objects that are dropped into the open top end. For example, a traveler may use it to carry toiletries or other small items while on a long flight or train or bus ride.

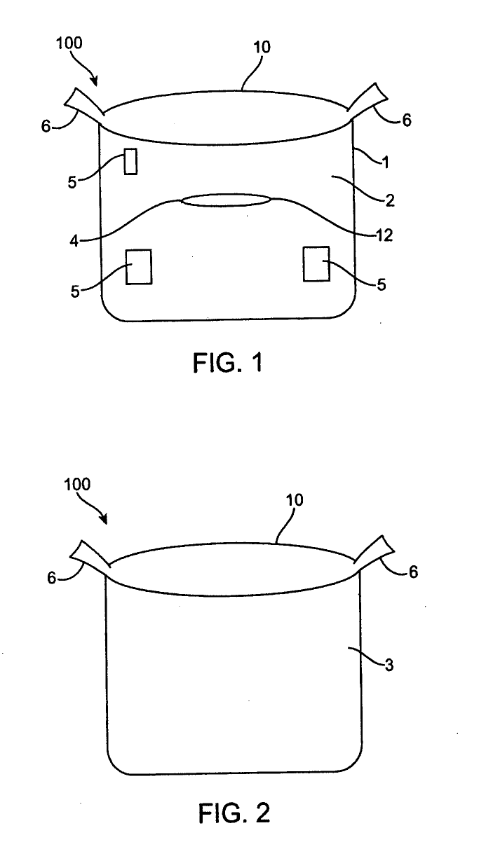

Turning now to the attached figures, an exemplary embodiment is illustrated in FIGS. 1 and 2. The arm support device 100 includes a sack 1 of flexible and stretchable material. The sack 1 is dimensioned such that, when completely unstretched, it is approximately as wide as the shoulders of the intended user. Additionally, it is shorter in length than the intended user's arms. Finally, its perimeter is approximately equal to the perimeter of the intended user's torso measured at about shoulder level and including the shoulders or upper arms in the perimeter measurement. The shape factor of this embodiment of the arm support device 100 is about 1.05.

The sack 1 of the arm support device 100 has a front face 2 and a rear face 3, with a top edge 10 around the open top end. On the front face 2 is an optional small hole or slit 4 that is closeable with a fastener 12 which, in this embodiment, is a hook and loop fastener. Also on the front face 2 of the sack 1 are optional pockets 5 for holding eyeglasses, personal electronic devices, pencils, and the like. The slit 4 allows the user to reach out of the sack 1 to access the pockets 5 or nearby objects. On the inside of the sack 1 near the top edge 10 are two optional pull tabs 6 that aid the user in pulling the sack 1 over the upper arms.

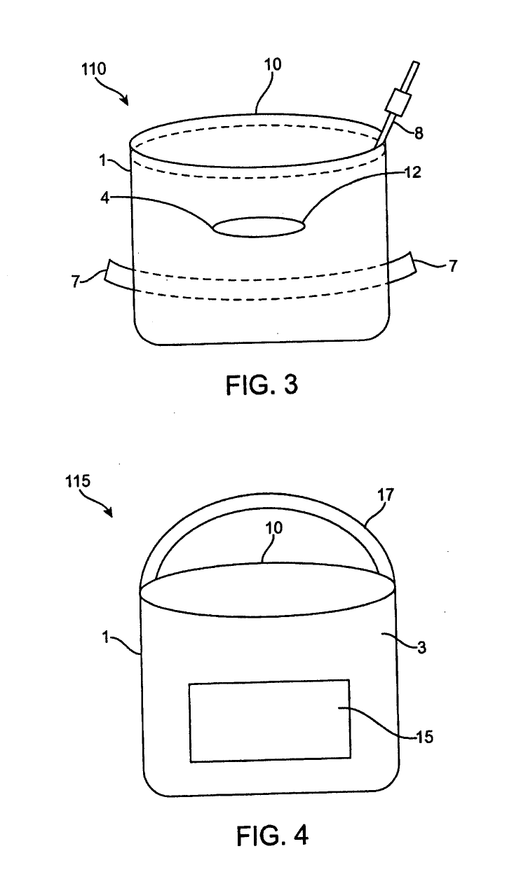

In another embodiment, as shown in FIG. 3, an arm support device 110 further includes optional straps 7 sewn into the sack to allow the amount of arm support provided by the sack 1 to be adjusted. Because the sack 1 of this embodiment has no head/neck opening that would cause items placed inside to fall out, it can be used as a travel bag. The top edge 10 of the sack 1 has an optional drawstring 8 around its perimeter to improve its functionality as a travel bag. In this embodiment, the only hole 4 in the sack 1 is on the front face 2 and it is closeable with a hook and loop fastener. With the hole 4 closed, items placed inside the arm support device will not fall out.

As shown in FIG. 4, another embodiment of an arm support 115 includes an optional pocket 15 on the rear face 3 into which the entire arm support 1 15 can be stuffed when not in use, such that it is self-contained to save space. Alternatively, this pocket 15 may be on the front face 2. The arm support 115 may further include an optional neck strap 17 with both ends attached to the top edge. The strap 17 provides additional vertical arm support for the user, and may also be used as a handle if the arm support device 115 is being used as a bag. Alternatively, the strap 17 may be several inches wide and stuffed with batting or other soft material so that the strap 17 can be used as a pillow when the arm support device 115 is worn.

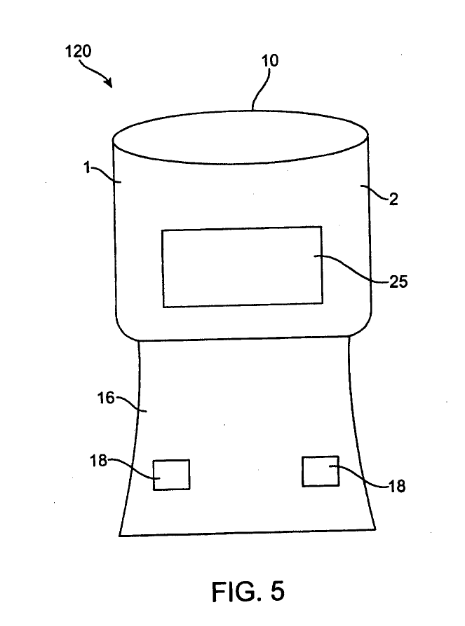

As shown in FIG. 5, another embodiment of an arm support device 120 includes an optional blanket 16 affixed to the bottom edge of the sack 1. Thus, a user can spread the blanket 16 over the user's legs to further increase the comfort provided. The blanket 16 may further include optional pockets 18. In this embodiment, the sack 1 may have an optional pocket 25 for storing the blanket 16 when not in use.

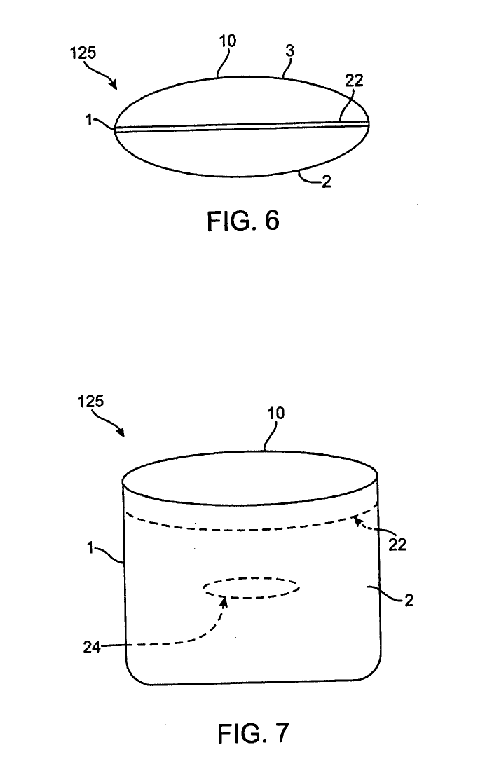

Another embodiment of the arm support device 125 is shown in FIGS. 6 and 7. The arm support device 125 includes an optional divider 22 within the sack 1, thus forming two interior compartments. The divider 22 allows the user to place one arm in the first compartment and the other arm in the second compartment. The divider 22 may include an optional slit 24 connecting the two compartments within the interior of the sack 1. The slit 24 may be closeable by fasteners such as hook and loop, buttons, zippers, or the like.

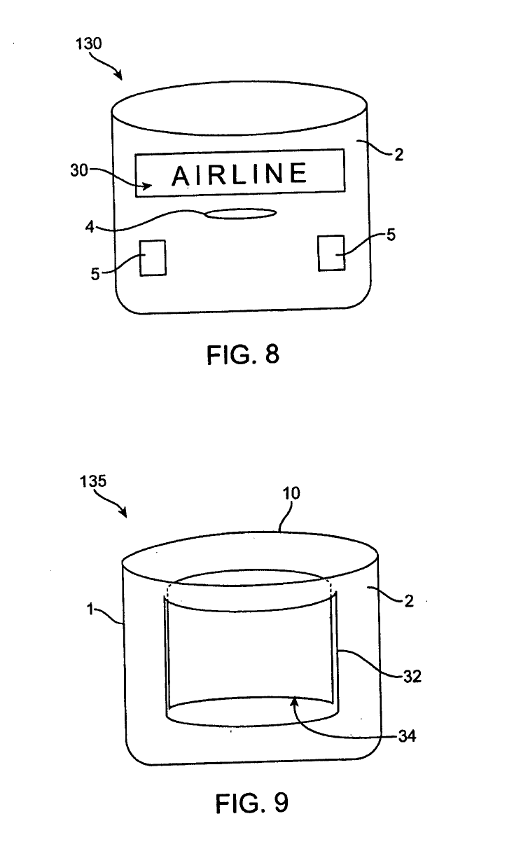

In another embodiment, as shown in FIG. 8, the arm support device 130 includes space on the front face 2 for optional indicia 30 such as text, advertising, graphic designs, or the like. For example, an airline may sell arm support devices 130 onboard long distance flights, and the arm support devices 130 may include the airline's logo on the front face 2.

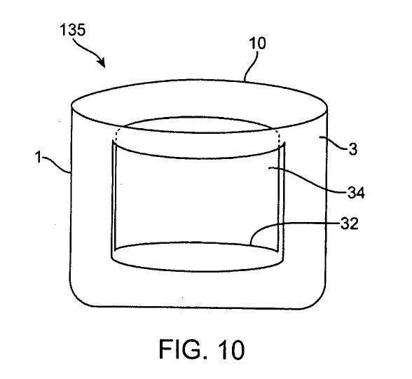

Yet another embodiment is illustrated in FIGS. 9 and 10, wherein the arm support device 135 has optional ventilation openings 32 and 34 on the front and/or rear faces respectively. The area of these openings may be nearly as large as the area of the faces on which they are located, or they may be much smaller. Larger openings would be used in arm support devices designed for warmer climates so that the user does not get too hot when using it.

Various modifications and alterations of the invention will become apparent to those skilled in the art without departing from the spirit and scope of the invention, which is defined by the accompanying claims. For example, it should be noted that steps recited in any method claims below do not necessarily need to be performed in the order they are recited. For example, in certain embodiments, steps may be performed simultaneously. The accompanying claims should be constructed with these principles in mind. Furthermore, any element in a claim that does not explicitly state “means for” performing a specified function or “step for” performing a specified function is not to be interpreted as a “means” or “step” clause as specified in 35 U.S.C. §112, ¶6.

1.) An arm support device comprising:
a sack of stretchable material having a front face, a rear face, an open top end, a top edge, and a closed bottom end,
the stretch factor of the perimeter of the top edge of the sack being between 1.25 and 2.0, and
the shape factor of the sack being between 0.5 and 2.0.

2.) The arm support device of claim 1 wherein the front face of the sack includes a slit.

3.) The arm support device of claim 2 wherein the slit is between 35% and 65% as wide as the unstretched width of the sack.

4.) The arm support device of claim 2 wherein a fastener is affixed to the edges of said slit, such that said slit is closeable.

5.) The arm support device of claim 4, wherein said fastener comprises a hook and loop fastener.

6.) The arm support device of claim 1 wherein the front face of the sack includes a pocket.

7.) The arm support device of claim 6 wherein said pocket is located on the external side of the front face.

8.) The arm support device of claim 6 wherein said pocket is located on the internal side of the front face.

9.) The arm support device of claim 1 further comprising a drawstring, wherein said drawstring is located inside the lining of said sack along the top edge of said sack, whereby the open top end of said sack is closeable by tightening said drawstring.

10.) The arm support device of claim 1 wherein said sack further comprises a pull tab attached to an internal surface of said sack near the top edge of said sack.

11.) The arm support device of claim 1 further comprising a blanket attached to an external surface of said sack.

12.) The arm support device of claim 11 wherein the blanket includes at least one pocket.

13.) The arm support device of claim 11 wherein the sack further includes a blanket storage pocket.

14.) The arm support device of claim 1 wherein the stretch factor of the length of said sack is between 1.1 and 1.3.

15.) The arm support device of claim 1 wherein the stretch factor of the length of said sack is approximately 1.2.

16.) The arm support device of claim 1 wherein the stretch factor of the perimeter of the top edge of said sack is approximately 1.5.

17.) The arm support device of claim 1 wherein the shape factor is between 0.8 and 1.3.

18.) The arm support device of claim 1 wherein the shape factor is 1.1.

19.) The arm support device of claim 1 further comprising a neck strap having two ends, both ends of said neck strap being affixed to the top edge of said sack.

20.) The arm support device of claim 19 wherein the neck strap is stuffed with batting.

21.) The arm support device of claim 1 wherein the front face of the sack includes indicia.

22.) The arm support device of claim wherein said bottom end is closed by the releasable engagement of fasteners affixed to said bottom end.

23.) A method of supporting the arms of a person, comprising:
a. placing a stretchable sack with a closed bottom end, an open top end, and a top edge over the arms of a person,
b. pulling the front-facing top edge over the person's shoulders but not over the person's head,
c. pulling the rear-facing top edge up to the person's underarm,
d. the person folding his or her arms across his or her torso,
e. the person relaxing his or her arm muscles such that the arms begin to fall out of the arms-folded position and begin to stretch the sack, and
f. the sack's material providing forces that oppose further stretching, said material supporting the person's arms from falling any further out of the arms-folded position.

24.) The method of claim 23 wherein the person uses pull tabs affixed to an inside surface of the sack to assist in pulling the front-facing top edge over the person's shoulders but not over the person's head, and to assist in pulling the rear-facing top edge up to the person's underarm.

25.) The method of claim 23 further comprising frictionally holding the sack in place on the user's upper arms.
